(19) United States
(12) Patent Application Publication
(10) Pub. No.: US 2026/0083909 A1
(43) Pub. Date: Mar. 26, 2026

(54) SYSTEM AND METHOD FOR CALCULATING AN INSULIN DOSING FUNCTION

(71) Applicant: University of Virginia Patent Foundation, Charlottesville, VA (US)

(72) Inventors: Anas El Fathi, Charlottesville, VA (US); Marc D. Breton, Charlottesville, VA (US); Elliott C. Pryor, Charlottesville, VA (US); Ali Tavasoli, Charlottesville, VA (US); Heman Shakeri, Charlottesville, VA (US)

(21) Appl. No.: 19/335,674

(22) Filed: Sep. 22, 2025

Related U.S. Application Data
(60) Provisional application No. 63/696,961, filed on Sep. 20, 2024.

(51) Int. Cl. A61M 5/172; G06N 3/092; G16H 20/17

(52) U.S. Cl. CPC A61M 5/1723; G06N 3/092; G16H 20/17; A61M 2230/201

(57) ABSTRACT

A reinforcement learning process with self attention is used for insulin dosing decisions in an automated medical system. The State-Action-Reward-Next State (SARS) sequence is used. The state represents the current condition, including recent continuous glucose monitoring readings, insulin doses, meal information, and potentially other relevant factors like time of day or physical activity levels. Based on this state, the agent takes an action by deciding on an insulin dose. It then receives a reward, a numerical value quantifying the quality of the action, based on resulting glucose levels and their proximity to the target range. This leads to a new state, and the process repeats. Through this iterative process, the algorithm updates the neural network weights, allowing the agent to learn which actions lead to better outcomes in different states.

### CROSS-REFERENCE TO RELATED APPLICATIONS

This application claims priority to and the benefit of U.S. provisional patent application No. 63/696,961, filed on Sep. 20, 2024, and System, Method, and Computer Readable Medium for Context-Aware Personalized Learning to Optimize Insulin Doses, the disclosure of which is hereby incorporated by reference herein in its entirety.

### STATEMENT OF GOVERNMENT RIGHTS

None.

### BACKGROUND

Insulin therapy is a critical component of diabetes management for over 100 million individuals worldwide. Among these, people with type 1 diabetes (T1D) face a unique and lifelong challenge. Due to their inability to produce insulin naturally, they must constantly calculate and administer insulin doses to maintain healthy blood glucose levels.

One of the most complex aspects of this management is calculating mealtime insulin doses. These doses need to precisely counterbalance the expected rise in blood glucose following a meal. This task requires a deep understanding of several factors: (i) The individual's insulin sensitivity, which can vary over time; (ii) The macronutrient composition of the meal, particularly carbohydrates; (iii) The timing of insulin administration relative to the meal; (iv) Current glucose levels and trends; (v) Recent physical activity and other factors affecting insulin needs.

Traditionally, people with T1D develop this understanding through a combination of intuition, trial and error, and past experiences, rather than relying solely on exact calculations. This approach, while often effective, can be imprecise and lead to suboptimal glucose control.

This disclosure utilizes raw data that is amenable to numerous kinds of mathematical and computer implemented methods of analysis. In some embodiments, artificial intelligence and machine learning techniques may be used in optional embodiments of this disclosure. Machine Learning (ML) and Artificial Intelligence (AI) systems are in widespread use in customer service, marketing, and other industries, including medicine and science. Machine learning is considered a subset of more general artificial intelligence operations, and AI endeavors may utilize numerous instances of machine learning to make decisions, predict outputs, and perform human-like intelligent operations. Machine learning protocols typically involve programming a model that instantiates an appropriate algorithm for a given computing environment and training the model on a particular data set or domain with known historical results. The results are generally known outputs of many combinations of parameter values that the algorithm accesses during training. The model uses numerous statistical and mathematical operations to learn how to make logical decisions and generate new outputs based on the historical training data. Machine learning (ML) includes, but is not limited to, a number of models such as neural networks, deep learning algorithms, support vector machines, data clustering, regression models, and Monte Carlo simulations. Other models may utilize linear regression, logistic regression, support vector machines, K-means clustering, classification models such as a binary classifier or a multi-class classifier, clustering models, anomaly detection, other supervised learning models, and even combinations of one or more machine language model types. Most of these take vectors of data as inputs.

The term “artificial intelligence,” therefore, includes any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is generally a subset of AI that enables a machine to acquire knowledge by extracting patterns from raw data.

The term “representation learning” may be used as a subset of machine learning that enables a machine to automatically discover representations needed for feature detection, prediction, or classification from raw data. Representation learning techniques include, but are not limited to, autoencoders.

The term “deep learning” may also be considered a subset of machine learning that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns a function that maps an input (also known as feature or features) to an output (also known as target or target) during training with an unlabeled data set. In a semi-supervised model, the model learns a function that maps an input (also known as feature or features) to an output (also known as target or target) during training with both labeled and unlabeled data.

Some machine learning models are designed for a specific data set or domain and are highly expert at handling the nuances within that narrow domain. It is with respect to these and other considerations that the various aspects of the present disclosure as described below are presented.

This disclosure combines algorithms deciphered by artificial intelligence and machine learning with currently known systems and models that gather data from a patient on a real time basis. Accordingly this disclosure can utilize sensors and medical equipment that improve a system's ability to diagnose and treat a patient.

Brackets with numerals therein refer to references cited the below disclosure.

### SUMMARY

Embodiments of this disclosure include a computer implemented method of estimating a universal function for calculating an insulin dose for a subject. The method includes using a computer having a processor connected to computer memory storing software to implement computer readable instructions that perform steps. The steps include retrieving raw data of sets having a number (N) of observations of glucose levels, insulin doses, and/or carbohydrate intake estimates collected from a population of subjects over a selected time period. The method applies the raw data to a reinforcement learning (RL) neural network having self-attention subroutines by performing additional steps. The additional steps may include pre-processing the raw data; segmenting the raw data with a sliding window function; saving, in the computer memory, a state matrix of the raw data by rearranging segmented raw data to align periodic events within the raw data, identified across the population, as a time series of neural network data; calculating an encoded state matrix by applying the state matrix to an encoder component of the RL neural network, wherein the encoder component applies at least one self-attention layer to the state matrix; passing the encoded state matrix to an actor component and a value component programmed as subroutines of the RL neural network, wherein the actor component defines a function to estimate a current bolus dose of insulin (B_{N}) that is a suggested action to take for a proposed carbohydrate intake (M_{N}); and wherein the value component assigns a qualitative value to the suggested action by calculating a reward function using a target glucose value and a calculated glucose value that will result from the suggested action. The method continues by iteratively evaluating the reward function to maximize the reward function; and selecting a suggested action corresponding to a maximum reward value as a recommended bolus dose of insulin.

### BRIEF DESCRIPTION OF THE DRAWINGS

Reference will now be made to the accompanying drawings, which are not necessarily drawn to scale.

FIG. 1A is a high level flow chart of an example method of utilizing machine learning processes to process digital raw data and map metabolic activity within a selected anatomy of a subject.

FIG. 1B is a flow chart of one embodiment of a computer implemented method that may be used according to this disclosure to map metabolic activity in a subject's body.

FIG. 2 is an overview of a computing environment capable of utilizing machine learning processes to process digital images and map metabolic activity within a selected anatomy of a subject

FIG. 3A is a computer architecture diagram showing a computing system capable of implementing aspects of the present disclosure in accordance with one or more embodiments.

FIG. 3B is a computer architecture diagram showing a networking environment that allows for data communication with a computing system capable of implementing aspects of the present disclosure in accordance with one or more embodiments.

FIG. 4 is a block diagram that illustrates a system 130 including a computer system 140 and the associated Internet 11 connection upon which an embodiment may be implemented.

FIG. 5 illustrates a system in which one or more embodiments of the disclosure can be implemented using a network, or portions of a network or computers. The present disclosure may be practiced with or without a network.

FIG. 6 illustrates an embodiment that includes, but is not limited thereto, a system, method, and computer readable medium that provides for utilizing machine learning processes to process digital images and map metabolic activity within a selected anatomy of a subject.

FIG. 7 is an illustrative example of a Reinforcement Learning Framework that can be used in accordance with this disclosure.

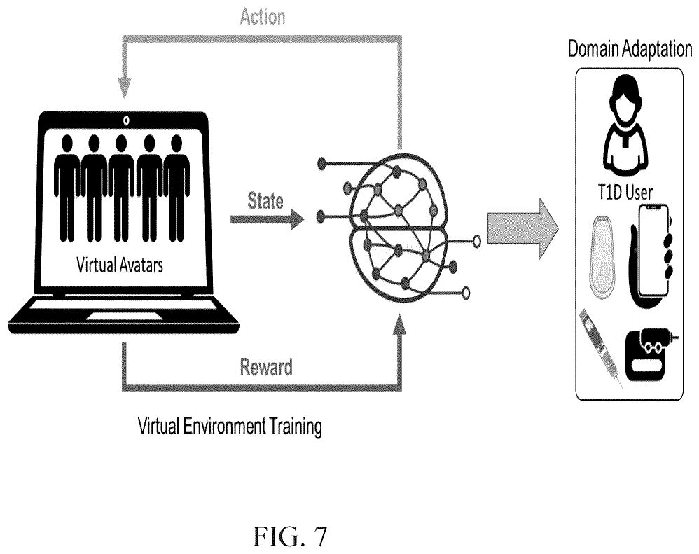

FIG. 8 is a schematic overview of steps of processing observations from patient monitoring in which: (i) data is normalized and decayed to emphasize the slow dynamics of insulin and meal, (ii) data is sliced using a window w and stride s, (iii) data is rearranged to align periodic events.

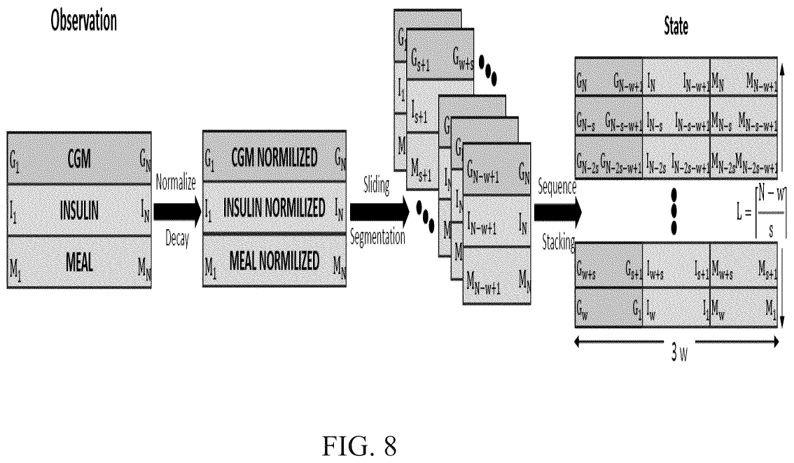

FIG. 9 is a graphical illustration depicting reward calculation in one embodiment of this disclosure. The smaller the shaded grey areas (1) and (2) the bigger the reward.

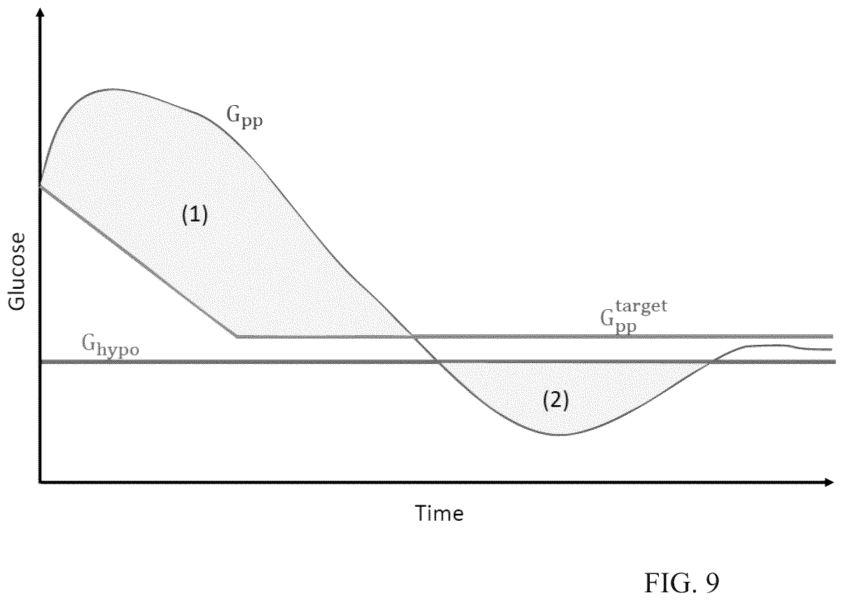

FIG. 10 is a schematic illustration of a neural network architecture including (1) the encoder network (specifically the self-attention network) (2) the value network (3) the actor network.

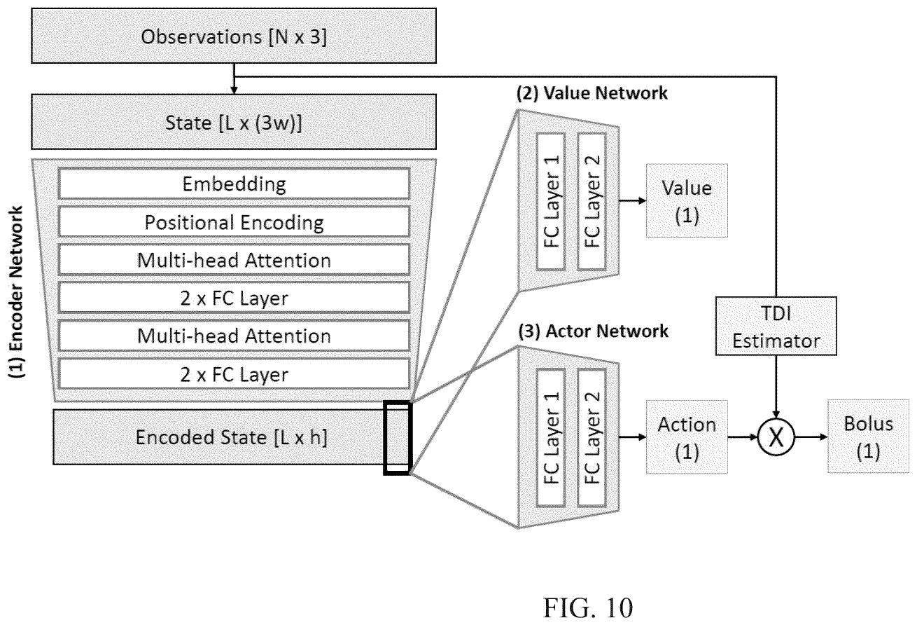

FIG. 11 is a graphical representation of in-silico results of embodiments herein with usual therapy (UT) or simplified therapy (ST) with aspects of this embodiment that uses insulin learning (IL) compared to usual therapy (UT) in (a) a sensor augmented pump (SAP) and (b) automated insulin delivery (AID) scenarios. In all scenarios, virtual subjects (VS) consumed random personalized meals, not all of which were announced to the bolus calculator, and meals had CC errors. In UT, a standard bolus calculator with personalized CR/ISF which is the carbohydrate ratio (CR) to insulin sensitivity factor (ISF) was used; In UT+IL the counted carbs were given to InsuLearn with at most 2 weeks of CGM/Insulin/meal information to calculate the bolus dose without CR/ISF information; In ST+IL only the information that a meal is to be consumed (0/1) is provided to InsuLearn. Results are 5-, 50-, 95-percentile of glucose, continuous glucose monitoring (CGM) and insulin outcomes of last 7 days of a 14-day simulation, repeated 6-times with different metabolic and behavioral variabilities of 20 virtual subjects (VS) (VS that were not used during training).

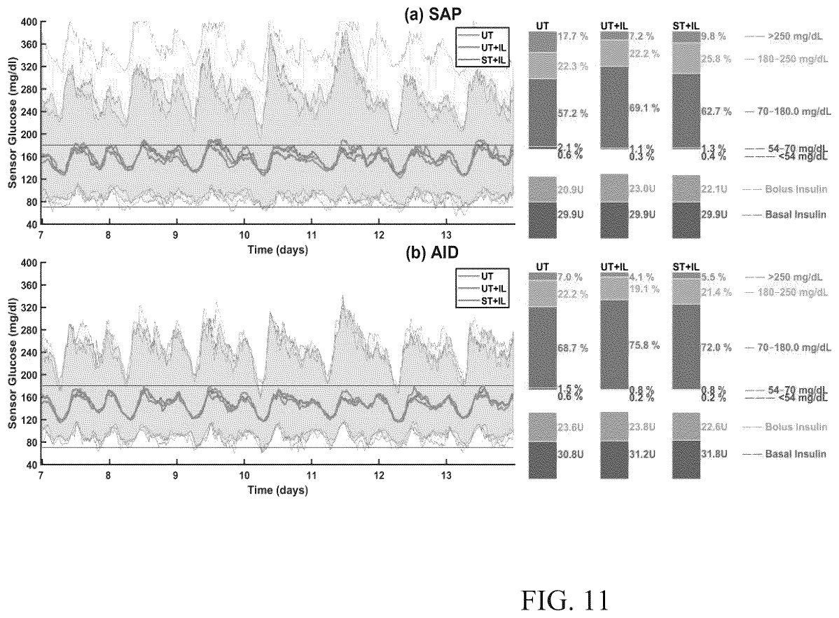

FIG. 12 are test results of the method used according to this disclosure.

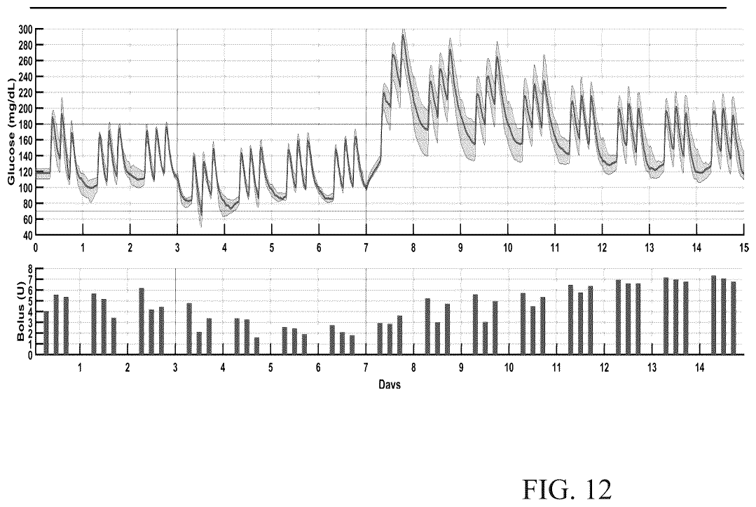

FIG. 13 is a Table 1 of parameters used in this disclosure.

FIG. 14 is a Table 2 comparing computer architectures used according to this disclosure.

### DETAILED DESCRIPTION

In some aspects, the disclosed technology relates to systems, methods, and computer-readable medium improving insulin therapy dosing. Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest.”

A detailed description of aspects of the disclosed technology, in accordance with various example embodiments, will now be provided with reference to the accompanying drawings. The drawings form a part hereof and show, by way of illustration, specific embodiments and examples. In referring to the drawings, like numerals represent like elements throughout the several figures.

An aspect of an embodiment of the present disclosure provides, among other things, a system, method and computer readable medium for providing a computer implemented paradigm that leverages the power of reinforcement learning (RL) to address challenges in insulin control for diabetes. Systems, methods, and products of this disclosure are designed to train a context-aware, personalized algorithm that can effectively mimic and enhance the insulin dosing process. Key features include adaptive learning in that the system can continuously learn from the individual's glucose responses, insulin doses, and meal data. This disclosure also provides for personalization for each patient by focusing on individual patterns and responses, i.e., this disclosure tailors its recommendations to each user's unique physiology. Context awareness: The algorithm considers various factors such as recent meals and insulin, and previous glucose trends to make more informed dosing decisions. Enhanced decision-making implemented herein includes analyzing patterns that may not be apparent to humans, but the computer implemented methods of this disclosure can potentially optimize insulin dosing beyond what is achievable through traditional methods. In many respects, this disclosure represents a significant step forward in diabetes management technology, offering the potential to improve glucose control, reduce the cognitive burden on individuals with Type One diabetes (T1D), and ultimately enhancing quality of life for millions of people living with this chronic condition.

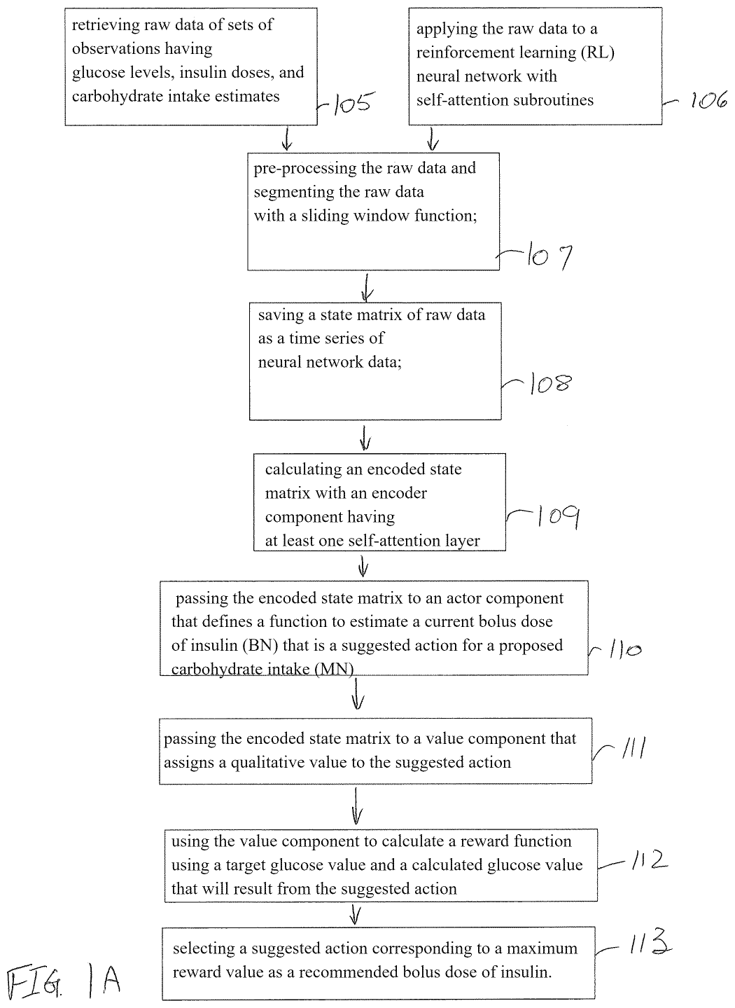

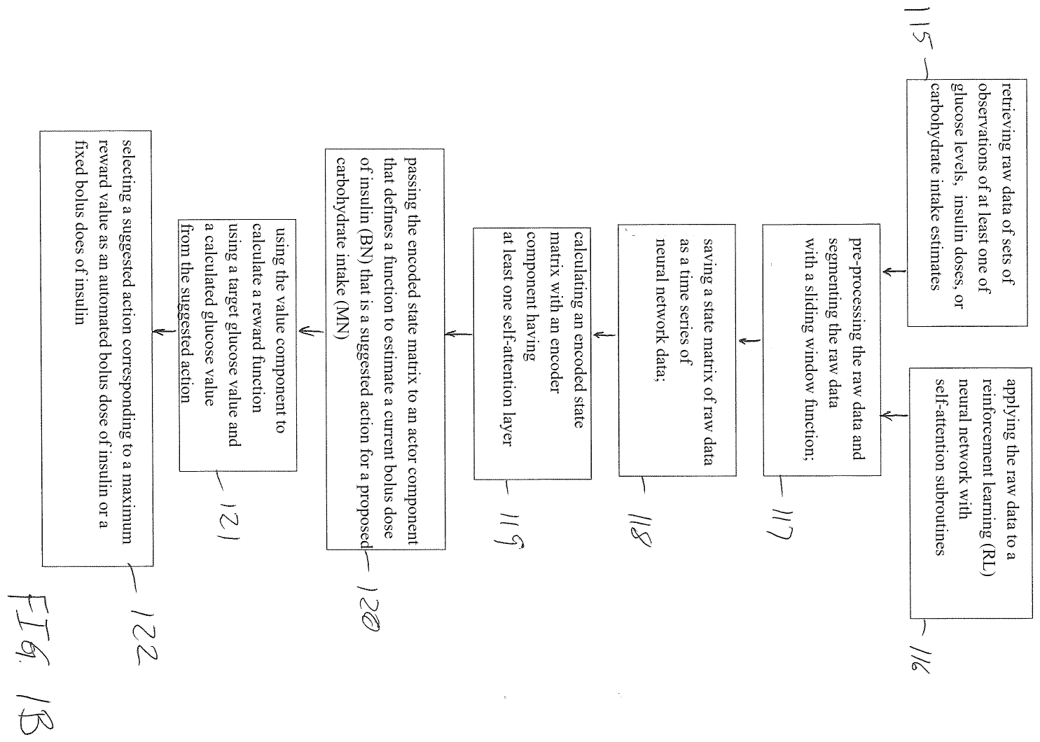

FIG. 1A illustrates a high level series of steps by which machine learning and artificial intelligence can be used to show how a neural network can be used to calculate insulin dosing as discussed herein.

FIG. 1B shows more details of a computerized method according to this disclosure in which a computer and associated software can prepare data for identifying raw data that can be selectively used to determine an insulin dose according to this disclosure.

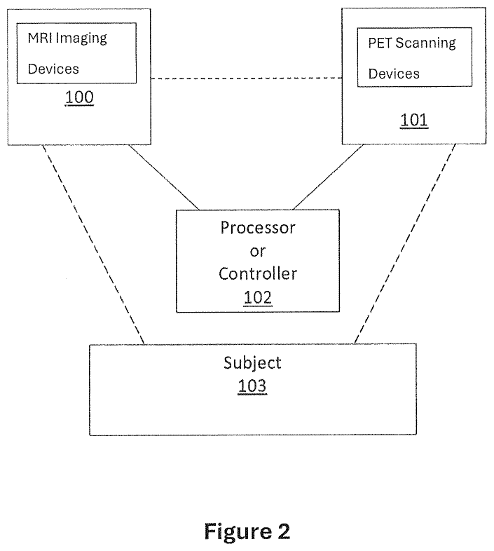

FIG. 2 is a high level functional block diagram of an embodiment of the present disclosure, or an aspect of an embodiment of the present disclosure. As shown in FIG. 2, a processor or controller 102 communicates with the glucose monitor or device 101, and optionally the insulin device 100. The glucose monitor or device 101 communicates with the subject 103 to monitor glucose levels of the subject 103. The processor or controller 102 is configured to perform the required calculations. Optionally, the insulin device 100 communicates with the subject 103 to deliver insulin to the subject 103. The processor or controller 102 is configured to perform the required calculations. The glucose monitor 101 and the insulin device 100 may be implemented as a separate device or as a single device. The processor 102 can be implemented locally in the glucose monitor 101, the insulin device 100, or a standalone device (or in any combination of two or more of the glucose monitor, insulin device, or a stand along device). The processor 102 or a portion of the system can be located remotely such that the device is operated as a telemedicine device. FIG. 2 also illustrates sensors and detectors that can be used to gather field data measurements for a subject, in real time or from samples, from the patient's blood. These kinds of sensors and detectors may be stand alone equipment or incorporated into an insulin delivery device or pump.

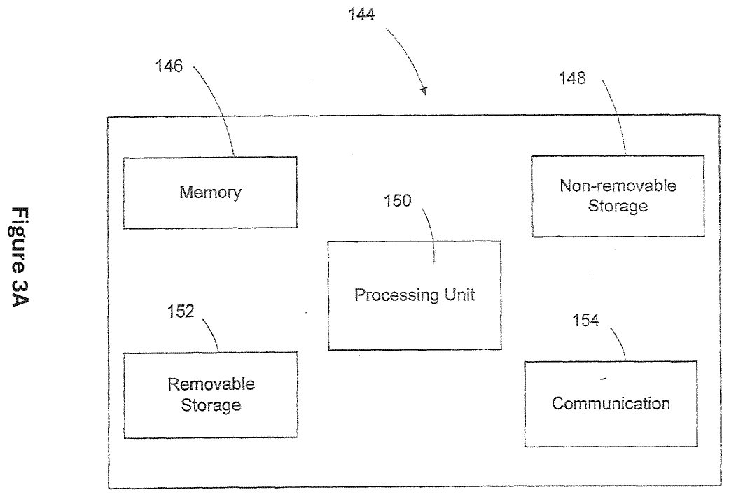

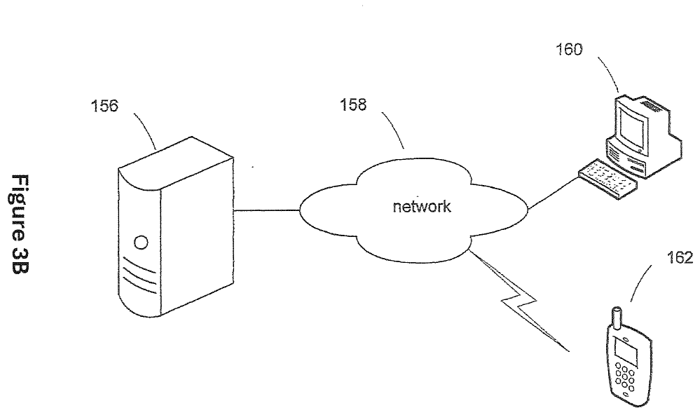

Referring to FIG. 3A, in its most basic configuration, computing device 144 typically includes at least one processing unit 150 and memory 146. Depending on the exact configuration and type of computing device, memory 146 can be volatile (such as RAM), non-volatile (such as ROM, flash memory, etc.) or some combination of the two.

Additionally, device 144 may also have other features and/or functionality. For example, the device could also include additional removable and/or non-removable storage including, but not limited to, magnetic or optical disks or tape, as well as writable electrical storage media. Such additional storage is the figure by removable storage 152 and non-removable storage 148. Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, the device.

The device may also contain one or more communications connections 154 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode, execute, or process information in the signal. By way of example, and not limitation, communication medium includes wired media such as a wired network or direct-wired connection, and wireless media such as radio, RF, infrared and other wireless media. As discussed above, the term computer readable media as used herein includes both storage media and communication media.

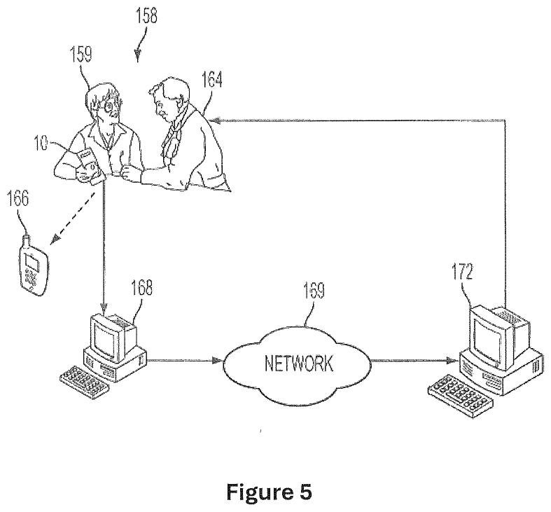

In addition to a stand-alone computing machine, embodiments of the disclosure can also be implemented on a network system comprising a plurality of computing devices that are in communication with a networking means, such as a network with an infrastructure or an ad hoc network. The network connection can be wired connections or wireless connections. As a way of example, FIG. 5 illustrates a network system in which embodiments of the disclosure can be implemented. In this example, the network system comprises computer 156 (e.g. a network server), network connection means 158 (e.g. wired and/or wireless connections), computer terminal 160, and PDA (e.g. a smart-phone) 162 (or other handheld or portable device, such as a cell phone, laptop computer, tablet computer, GPS receiver, mp3 player, handheld video player, pocket projector, etc. or handheld devices (or non portable devices) with combinations of such features). In an embodiment, it should be appreciated that the module listed as 156 may be glucose monitor device. In an embodiment, it should be appreciated that the module listed as 156 may be a glucose monitor device, artificial pancreas, and/or an insulin device (or other interventional or diagnostic device). Any of the components shown or discussed with FIG. 3B may be multiple in number. The embodiments of the disclosure can be implemented in anyone of the devices of the system. For example, execution of the instructions or other desired processing can be performed on the same computing device that is anyone of 156, 160, and 162. Alternatively, an embodiment of the disclosure can be performed on different computing devices of the network system. For example, certain desired or required processing or execution can be performed on one of the computing devices of the network (e.g. server 156 and/or glucose monitor device), whereas other processing and execution of the instruction can be performed at another computing device (e.g. terminal 160) of the network system, or vice versa. In fact, certain processing or execution can be performed at one computing device (e.g. server 156 and/or insulin device, artificial pancreas, or glucose monitor device (or other interventional or diagnostic device)); and the other processing or execution of the instructions can be performed at different computing devices that may or may not be networked. For example, the certain processing can be performed at terminal 160, while the other processing or instructions are passed to device 162 where the instructions are executed. This scenario may be of particular value especially when the PDA 162 device, for example, accesses to the network through computer terminal 160 (or an access point in an ad hoc network). For another example, software to be protected can be executed, encoded or processed with one or more embodiments of the disclosure. The processed, encoded or executed software can then be distributed to customers. The distribution can be in a form of storage media (e.g. disk) or electronic copy.

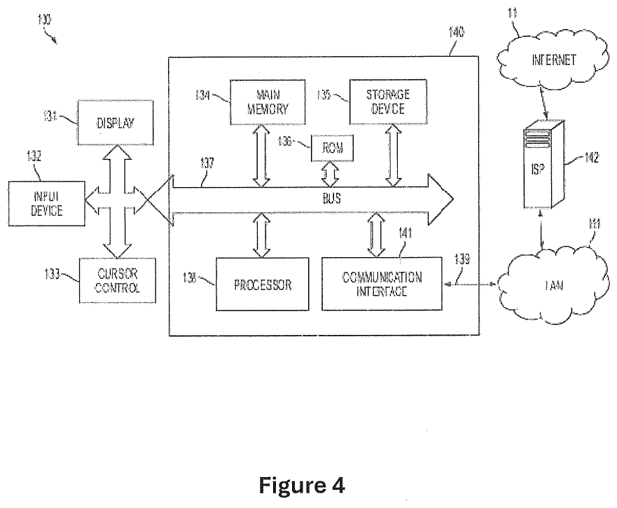

FIG. 4 is a block diagram that illustrates a system 130 including a computer system 140 and the associated Internet 11 connection upon which an embodiment may be implemented. Such configuration is typically used for computers (hosts) connected to the Internet 11 and executing a server or a client (or a combination) software. A source computer such as laptop, an ultimate destination computer and relay servers, for example, as well as any computer or processor described herein, may use the computer system configuration and the Internet connection shown in FIG. 4. The system 140 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 4 illustrates various components of a computer system, it is not intended to represent any particular architecture or manner of interconnecting the components; as such details are not germane to the present disclosure. It will also be appreciated that network computers, handheld computers, cell phones and other data processing systems which have fewer components or perhaps more components may also be used. The computer system of FIG. 4 may, for example, be an Apple Macintosh computer or Power Book, or an IBM compatible PC. Computer system 140 includes a bus 137, an interconnect, or other communication mechanism for communicating information, and a processor 138, commonly in the form of an integrated circuit, coupled with bus 137 for processing information and for executing the computer executable instructions. Computer system 140 also includes a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 further includes a Read Only Memory (ROM) 136 (or other non-volatile memory) or other static storage device coupled to bus 137 for storing static information and instructions for processor 138. A storage device 135, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically computer system 140 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 132, including alphanumeric and other keys, is coupled to bus 137 for communicating information and command selections to processor 138. Another type of user input device is cursor control 133, such as a mouse, a trackball, or cursor direction keys for communicating direction information and command selections to processor 138 and for controlling cursor movement on display 131. This input device typically has two degrees of freedom in two axes, a first axis (e.g., x) and a second axis (e.g., y), that allows the device to specify positions in a plane.

The computer system 140 may be used for implementing the methods and techniques described herein. According to one embodiment, those methods and techniques are performed by computer system 140 in response to processor 138 executing one or more sequences of one or more instructions contained in main memory 134. Such instructions may be read into main memory 134 from another computer-readable medium, such as storage device 135. Execution of the sequences of instructions contained in main memory 134 causes processor 138 to perform the process steps described herein. In alternative embodiments, hard-wired circuitry may be used in place of or in combination with software instructions to implement the arrangement. Thus, embodiments of the disclosure are not limited to any specific combination of hardware circuitry and software.

The term “computer-readable medium” (or “machine-readable medium”) as used herein is an extensible term that refers to any medium or any memory, that participates in providing instructions to a processor, (such as processor 138) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 137. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 138 for execution. For example, the instructions may initially be carried on a magnetic disk of a remote computer. The remote computer can load the instructions into its dynamic memory and send the instructions over a telephone line using a modem. A modem local to computer system 140 can receive the data on the telephone line and use an infra-red transmitter to convert the data to an infra-red signal. An infra-red detector can receive the data carried in the infra-red signal and appropriate circuitry can place the data on bus 137. Bus 137 carries the data to main memory 134, from which processor 138 retrieves and executes the instructions. The instructions received by main memory 134 may optionally be stored on storage device 135 either before or after execution by processor 138.

Computer system 140 also includes a communication interface 141 coupled to bus 137. Communication interface 141 provides a two-way data communication coupling to a network link 139 that is connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 141 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (02-20-04), which is incorporated in its entirety for all purposes as if fully set forth herein.

Wireless links may also be implemented. FIG. 5 illustrates setups 158 in which multiple parties 159, 164 share information across a network 169 with numerous devices that can be a handheld telephone or mobile device 10, 166 or standard computers 168, 172. In any such implementation, communication interface 141 sends and receives electrical, electromagnetic or optical signals that carry digital data streams representing various types of information. Network link 139 typically provides data communication through one or more networks to other data devices. For example, network link 139 may provide a connection through local network 111 to a host computer or to data equipment operated by an Internet Service Provider (ISP) 142. ISP 142 in turn provides data communication services through the world-wide packet data communication network Internet 11. Local network 111 and Internet 11 both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

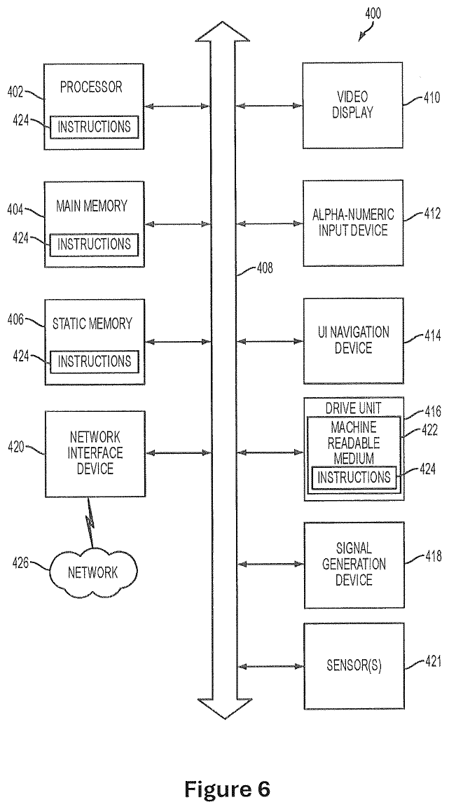

FIG. 6 is a block diagram illustrating an example of a machine upon which one or more aspects of embodiments of the present disclosure can be implemented.

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 410, input device 412 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

This disclosure represents a significant advancement in diabetes management technology, utilizing state-of-the-art machine learning techniques to optimize insulin dosing. This disclosure integrates a sophisticated neural network-based agent that performs complex analysis of multi-modal time series data from sources, including but not limited to Continuous Glucose Monitor (CGM) readings, insulin administration records, an meal consumption data, such as carbohydrate intake estimates. Systems, methods and products discussed herein include an ability to identify and learn from individual behavioral and physiological patterns, creating a highly personalized approach to insulin dosing optimization. Non-limiting embodiments of this disclosure employ a transformer architecture, which has shown remarkable success in processing sequential data across various domains, particularly in natural language processing. This choice of architecture is significant for several reasons discussed herein.

Self-attention mechanisms: This disclosure utilizes self attention components used in neural networks as discussed in the article by Vaswani, et al., Attention is All You Need arXiv:1706.03762v7 [cs.CL]2 Aug. 2023, which is incorporated by reference as if set forth fully herein. As discussed by Vaswani, “[a]n attention function can be described as mapping a query and a set of key-value pairs to an output, of the values, where the weight assigned to each value is computed by a compatibility function of the query with the corresponding key where the query, keys, values, and output are all vectors. The output is computed as a weighted sum of the values, where the weight assigned to each value is computed by a compatibility function of the query with the corresponding key . . . .” The two most commonly used attention functions are additive attention and dot-product (multiplicative) attention. These allow the model to weigh the importance of different elements in the input sequence dynamically. In the context of diabetes management, this means the system can learn to focus on the most relevant past events or data points when making dosing decisions. This disclosure also uses context-aware encoding by processing extended time series data, the model can contextualize recent inputs (CGM readings, insulin doses, meals) within a broader historical context. This enables the system to capture long-term dependencies and cyclical patterns that may be crucial for accurate insulin dosing.

The following sections detail this disclosure's architecture and training process, explaining how the system enhances its predictive capabilities through reinforcement learning.

### Reinforcement Learning Framework

This disclosure agent leverages RL to optimize insulin dosing decisions, a crucial framework for developing a system that can adapt to the complex, dynamic environment of diabetes management. The agent undergoes initial training in a virtual environment using computer simulations, employing the validated diabetes simulator UVA/Padova T1D Simulator. This approach allows for safe, accelerated learning without risking patient safety, incorporating various factors like meal sizes, insulin absorption rates, and physiological variations to mimic real-world scenarios.

At the core of the RL process is the State-Action-Reward-Next State (SARS) sequence. The state represents the current condition, including recent CGM readings, insulin doses, meal information, and potentially other relevant factors like time of day or physical activity levels. Based on this state, the agent takes an action by deciding on an insulin dose. It then receives a reward, a numerical value quantifying the quality of the action, based on resulting glucose levels and their proximity to the target range. This leads to a new state, and the process repeats. Through this iterative process, the RL algorithm updates the neural network weights, allowing the agent to learn which actions lead to better outcomes in different states.

After initial training, the agent develops a generalized policy applicable to a broad population, essentially creating a sophisticated, context-aware insulin dosing strategy. To tailor the model to individual patients, a domain adaptation process is employed, involving fine-tuning the pre-trained model using patient-specific data. This allows the system to adapt to individual physiological responses and behaviors (FIG. 1).

This comprehensive RL framework enables This disclosure to learn complex insulin dosing strategies that can adapt to individual patients and changing conditions. The use of PPO and domain adaptation techniques suggests a robust approach that balances generalization with personalization, potentially leading to improved glucose control and reduced patient burden.

### Proximal Policy Optimization

This disclosure system employs Proximal Policy Optimization (PPO), an advanced online RL algorithm known for its stability and efficiency (Schulman et al., 2017). PPO's key features include on-policy learning, where the agent learns from its own recent experiences, and trust region optimization, which limits the size of policy updates to prevent catastrophic forgetting. It also uses a clipped objective function, helping to avoid excessively large policy updates.

Proximal Policy Optimization (PPO) is a state-of-the-art deep reinforcement learning algorithm. Policy gradient-based methods (like PPO) have been shown to be very effective in high-dimensional problems with continuous action spaces. PPO is an on-policy learning algorithm that makes small, constrained steps from the current policy through a clipped objective function. The loss function for PPO is given in the following equation:

$$L ( θ ) = E [ min ( {r}_{t ( θ ) {A}_{t}} , clip ( {r}_{t ( θ )} , 1 - ϵ , 1 + ϵ ) {A}_{t} ) ]$$

$$where {r}_{t} ( θ ) = \frac{π ( {a}_{t} \vert {s}_{t} )}{{π}_{o l d} ( {a}_{t} \vert {s}_{t} )}$$

is the ratio of the probabilities of taking the action of the current policy, divided by the probability of taking that action under the previous policy.

If rₜ>1 then the action becomes more likely under the new policy. At is the advantage term that defines the amount of reward (estimated via the Bellman equation) this action gives relative to the average value.

The primary loss term rₜ*Aₜ intuitively means that if the advantage is positive, we want to make that action more likely, and if the advantage is negative, make the action less likely. The intuition of the clipping term is that the approximation of the policy gradient is only valid near the old policy, so the loss is clipped to prevent large changes in the policy each iteration.

### State Formulation

The state formulation involves preprocessing and encoding of time-series data from CGM, insulin, and meal records, and includes derived features such as normalized glucose, insulin on board, and estimated meal absorption. State is defined as a sequence of CGM/Insulin/Meal information data in the past T minutes. At a time tₙ, we assume the availability of historical data at a fixed sampling time

$${t}_{s} = \frac{T}{N} : ( i ) CGM :$$

measured glucose {\tilde{G}ₖ}_{k∈1:N}, (ii) basal and bolus insulin {Iₖ}_{k∈1:N}, the basal insulin is converted to amounts of insulin units delivered during the sampling tₛ, (iii) estimated amount of carbohydrates in meals {Mₖ}_{k∈1:N}.

Because of the asymmetric relevance of low glucose values compared to high glucose values, we normalize the CGM by a log-transform as shown in equation

$$G = \frac{2 log ( \tilde{G} ) - ( log + log )}{log - log}$$

$$where = 180 and = 70$$

are chosen to match hyper-/hypo-glycemic levels to 1 and −1, respectively.

We model the delays in insulin and meal absorption using the following generic decay function.

$$d ( k , τ , {t}_{s} ) = ( 1 + \frac{( k - 1 ) {t}_{s}}{T} ) {e}^{- \frac{( k - 1 ) {t}_{s}}{τ}}$$

where τ is a time constant related to the time-to-maximum effects.

The insulin and meal information are normalized using the decay function d(.) and the sum of all events during the 14 days:

$$\begin{matrix}{I}_{k} = \frac{{\sum }_{j = 1}^{k} d ( k - j + 1 , {τ}_{i} , {t}_{s} )}{{\sum }_{i = 1}^{N} {\sum }_{j = 1}^{k} d ( i - j + 1 , {τ}_{i} , {t}_{s} )} \\ {M}_{k} = {α}_{m} \frac{{\sum }_{j = 1}^{k} d ( k - j + 1 , {τ}_{m} , {t}_{s} )}{{\sum }_{i = 1}^{N} {\sum }_{j = 1}^{i} d ( i - j + 1 , {τ}_{m} , {t}_{s} )}\end{matrix}$$

where τᵢ=75 minutes and τₘ=45 minutes and αₘ=¼ is a scaling factor that represents the inverse of the average number of meals per day.

Due to the length of the observed data, the sparsity of the insulin bolus information, and the local correlation of cgm/insulin/meals, we choose to further transform the observation, as shown in FIG. 2. First, we perform a sliding window segmentation of the time series with width w and overlap s. The data is then rearranged as a new sequence where each sequence element is an array of size 3*w containing related glucose/insulin/meal data. The new sequence, called state, is of length

$$L = ⌈ \frac{{Nt}_{s} - w}{s} ⌉ .$$

We selected w=240 minutes and s=120 minutes, to achieve a reasonable shrinkage of the observation L=166.

### Reward Formulation

The reward formulation is crucial for guiding the learning process. It's a composite function considering factors such as time in target glucose range, frequency and severity of hypoglycemic and hyperglycemic events, glucose variability, and proximity to individualized glucose targets.

Following each action, the next 8 hours of glucose is extracted to estimate the reward of the action. If another action occurs within 8 hours, only the glucose before the next action is considered. We require a minimum of 3 hours of glucose data for the action to be considered for training. This signal is referred to as Gₚₚ. The reward R is calculated using Gₚₚ as indicated in equation X.

$$R = - \frac{\frac{1}{\# {G}_{pp}} \sum max ( {G}_{pp} - {G}_{pp}^{target} , 0 ) - {μ}_{iAUC}}{{σ}_{iAUC}} - \frac{10}{\# {G}_{pp}} \sum Risk ( {G}_{pp} ) ( {G}_{pp} < {G}_{hypo} )$$

$$where {G}_{pp}^{target}$$

is a trace connecting the glucose levels at time of the action to the desired glucose target (110 mg/dL) using a slope of 20 mg/dL/h, G_{hypo}=90 mg/dL is a threshold to penalize lower glucose levels. Gₚₚ stands for the size of the Gₚₚ array. Risk(Gₚₚ)=log Gₚₚ^{1.084}−5.381 is the risk function as defined by (Kovatchev, 2017). μ_{iAUC}=60 mg/dL and σ_{iAUC}=45 mg/dL are tuning parameters representing the mean and standard deviation incremental area under the curve (iAUC) of glucose. FIG. 3 shows a visualization of the reward.

### Network Architecture and Action Formulation

In PPO, two neural networks are trained, a value and actor network. In This disclosure, both networks share most of weights to constitute an encoded state that is given to value branch and an actor branch. In one implementation, the encoding is implemented with a sequence of transformers. In another implementation, the encoding is implemented with a bi-LSTM architecture

The transformer architecture enables This disclosure to detect complex patterns related to:

Insulin sensitivity variations: Both short-term (e.g., due to physical activity) and long-term (e.g., due to hormonal changes).

Carbohydrate intake impact: Learning how different types and amounts of carbohydrates affect glucose levels.

Temporal patterns: Identifying time-of-day or day-of-week effects on insulin needs.

Inter-factor interactions: Understanding how combinations of factors (e.g., stress, illness, menstrual cycles) influence insulin requirements.

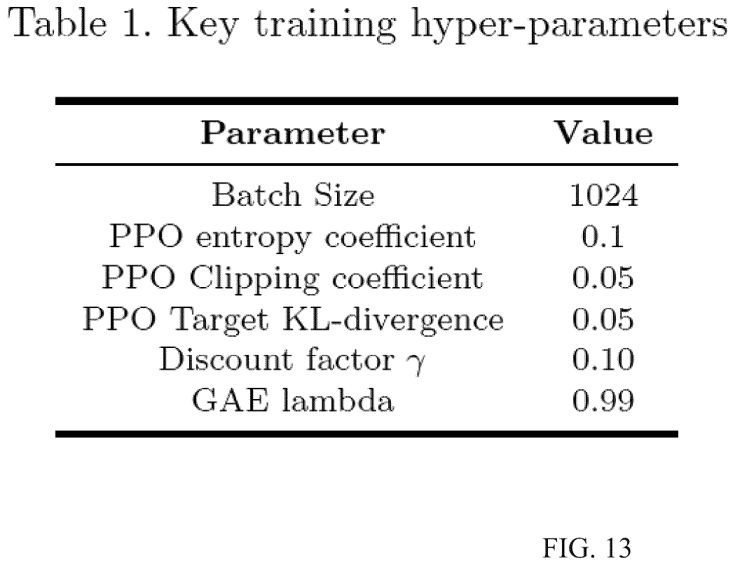

The system's architecture is designed to enhance predictive capabilities using parameters of FIG. 13, likely including:
a) Short-term glucose prediction: Anticipating glucose levels in the near future to inform immediate insulin dosing decisions.
b) Long-term trend analysis: Identifying patterns that may inform adjustments to overall insulin regimens.
c) Personalized risk assessment: Evaluating the likelihood of hypoglycemic or hyperglycemic events based on current conditions and historical patterns.

The action of the agent a_{N} is defined as a fraction of the estimated total daily insulin (TDI) (B_{N}=a_{N}×TDI). TDI is calculated directly from the observations as the total sum of insulin records per day. The action (the output of the actor-network) is bounded using a tangent hyperbolic transformation. FIG. 4 shows the architecture.

### Applications

### Bolus Calculator

This disclosure can be used to train a bolus calculator for people with diabetes using short/rapid acting insulin in a multiple daily injection or insulin pump therapy. This disclosure can work without the need of therapy parameters and can be used with full carbohydrate counts, or only by specifying meal categories instead of full carbohydrate counting.

For this application, the training and validation scripts were developed in Python 3.9, using PyTorch 2.1 library to implement the networks. The agent, comprising the encoder and actor networks, is serialized using TorchScript for efficiency and compatibility. We employed a C++ version of the FDA-recognized UVA/Padova T1D simulator, focusing on adult virtual subjects (VS) for in-silico experimentation. The PyTorch C++ API facilitated the integration of the agent within the simulator.

During the training, 80 VS were utilized. The simulations covered both sensor-augmented insulin pump (SAP) therapy, encompassing basal insulin, meal-accompanying bolus doses, and occasional correction doses for high glucose, and an automated insulin delivery (AID) system, specifically a legacy version of Control-IQ (Brown et al., 2019).

In each training epoch, 20 VS were randomly chosen for a 21-day simulation under both sensor-augmented-pump (SAP) and AID conditions, with two initial random seeds (yielding a total of 20×2×2=80 simulations). These seeds introduced variability in several aspects, such as wake-up times, meal timings and sizes, errors in therapy parameters, meal announcement inaccuracies, unanticipated eating activities, meal omissions, insulin dosing delays, and interday/intraday insulin sensitivity fluctuations. The agent, whose parameters were fixed during these simulations, determined the insulin bolus for each reported meal via a stochastic policy. The resulting 80-episode simulations are then processed to extract a sequence of transitions (state, actions, rewards, end states) that are used in training.

A total of 30 agents were trained, using five seeds across six distinct architectural designs for the encoder network. This included two attention networks (ATT) with varying parameter counts (250K and 70K), a bi-directional LSTM, and a standard LSTM both sized similarly to the larger ATT (250K), a larger biLSTM to account for double the hidden states (500K), and a simple fully connected (FC) network with parameters comparable to the smaller ATT (70K).

For validation, the remaining twenty VS underwent 14-day simulations under both SAP and AID settings, with three random seeds across two scenarios (20×2×3\times2=240 simulations). Scenario 1 replicated the training environment, introducing metabolic and behavioral variabilities, while Scenario 2 maintained only the variability in insulin sensitivity, representing an idealized condition where therapy parameters are perfectly known. The performance of the trained agents was evaluated against a standard bolus calculator to establish a baseline comparison.

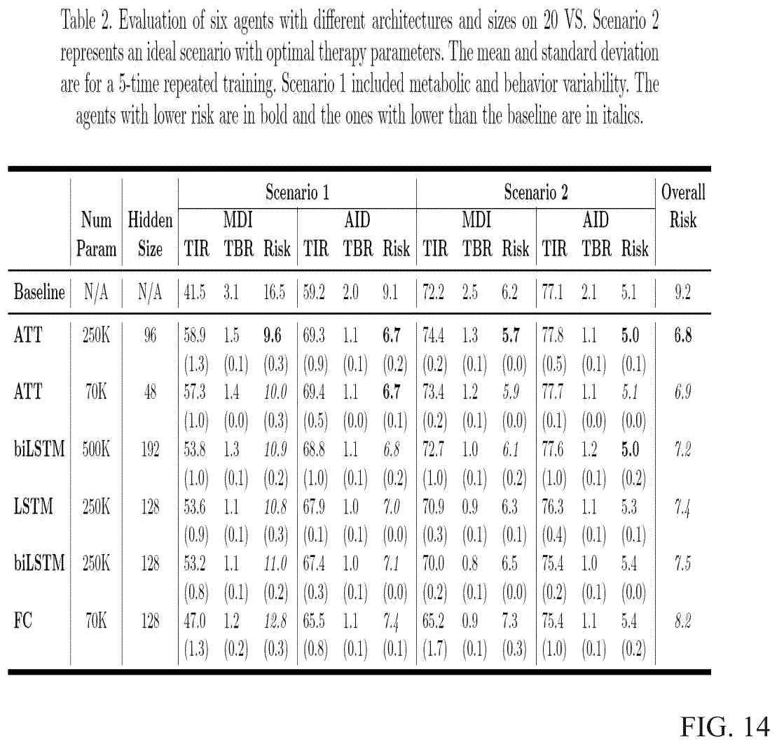

Table 2 of FIG. 14 summarizes the obtained results. Notably ATT-based encoder networks resulted in the smallest overall glycemic risk while requiring fewer parameters. The best ATT 250K network reduced risk in all scenarios and all therapy modalities. All the trained agents outperformed the baseline in the worst-case scenario 1 while not all agents were able to match the ideal scenario 2.

To further investigate the robustness of the best trained network we evaluated the ATT 250K network in an experiment using a simple meal announcement paradigm (a 0/1 indicator) rather than providing full meal information in both an AID and SAP scenario. Results are presented in

### Bolus Priming System

A bolus priming system is an automatic bolus dosing system that accompanies a fully closed system (an AID not requiring carbohydrate counting). This disclosure can be trained with only the CGM and insulin information (no meal information) to detect and predict upcoming meals.

### Closed-Loop System

Another application is to use the same architecture to directly train a closed-loop.

Embodiments of this disclosure include a computer implemented method of estimating a universal function for calculating an insulin dose for a subject. The method can be shown in FIG. 1A as using a computer comprising a processor connected to computer memory storing software to implement computer readable instructions that perform steps including retrieving raw data of sets (105) comprising a number (N) of observations comprising glucose levels, insulin doses, and carbohydrate intake estimates collected from a population of subjects over a selected time period; applying the raw data to a reinforcement learning (RL) neural network (106) comprising self-attention subroutines by performing additional steps including pre-processing the raw data (107); segmenting the raw data (108) with a sliding window function (109); saving, in the computer memory, a state matrix (110) nof the raw data by rearranging segmented raw data to align periodic events within the raw data, identified across the population, as a time series of neural network data; calculating an encoded state matrix by applying the state matrix to an encoder component of the RL neural network, wherein the encoder component applies at least one self-attention layer to the state matrix; passing the encoded state matrix (111) to an actor component and a value component programmed as subroutines of the RL neural network; wherein the actor component defines a function to estimate a current bolus dose of insulin (B_{N}) that is a suggested action to take for a proposed carbohydrate intake (M_{N}); and wherein the value component (112) assigns a qualitative value to the suggested action by calculating a reward function using a target glucose value and a calculated glucose value that will result from the suggested action; iteratively evaluating the reward function to maximize the reward function; and selecting a suggested action (113) corresponding to a maximum reward value as a recommended bolus dose of insulin.

In another embodiment, the pre-processing includes normalizing the raw data and/or applying a decay function to the raw data. In another embodiment, applying at least one self-attention layer to the state matrix includes saving a last hidden state matrix from the encoder component as the encoded state matrix. Segmenting the raw data may include saving slices of the data, wherein the slices of the data comprise multiple observations from the raw data corresponding to a window size and a stride size used to segment the raw data. The slices of the data may include related observations selected from glucose levels, insulin doses, or carbohydrate intake estimates. Respective slices of related observations are matched as sequence elements, and the sequence elements are combined into a sequence of length (L) having rows that include sequence elements having the related observations selected from glucose levels, insulin doses, or carbohydrate intake estimates. The actor component calculates the function, with the sequence in the state matrix of N observations over the time period using

B_{N}=f(M^{˜}_{N}, G^{˜}_{N}, H_{1:N-1}), wherein H_{1:N} is the complete data set of the encoded state matrix. The complete data set H_{1:N} includes insulin I equal to B_{N}+U_{N}, where B_{N} is an agent suggested action and U_{N} is any additional insulin delivered at t_{N}, including the delivered basal insulin.

The method calculates a universal function for calculating an insulin dose for a subject, with the method including using a computer having a processor connected to computer memory storing software to implement computer readable instructions that perform steps including retrieving raw data of sets comprising a number (N) of observations (115) comprising glucose levels and at least one of insulin doses or carbohydrate intake estimates collected from a population of subjects over a selected time period and applying the raw data (116) to a reinforcement learning (RL) neural network comprising self-attention subroutines by performing additional steps. The additional steps include pre-processing the raw data (117); segmenting the raw data with a sliding window function; saving, in the computer memory, a state matrix (118) of the raw data by rearranging segmented raw data to align periodic events within the raw data, identified across the population, as a time series of neural network data; calculating an encoded state matrix (119) by applying the state matrix to an encoder component of the RL neural network, wherein the encoder component applies at least one self-attention layer to the state matrix; passing the encoded state matrix to an actor component (119) and a value component programmed as subroutines of the RL neural network; wherein the actor component defines a function to estimate a current bolus dose of insulin (B_{N}) that is a suggested action (120) to take for a subject; wherein the value component (121) assigns a qualitative value to the suggested action by calculating a reward function using a target glucose value and a calculated glucose value that will result from the suggested action; iteratively evaluating the reward function to maximize the reward function; and selecting a suggested action corresponding to a maximum reward value (122) as a recommended bolus dose of insulin.

In one example, the raw data sets include glucose levels and insulin doses in the absence of carbohydrate intake estimates, and the suggested action is application of an automated bolus that delivers selected boluses of additional insulin to compensate for glucose increases.

In another example, the raw data sets include glucose levels in the absence of insulin doses and carbohydrate intake estimates, and the suggested action is application of an automated bolus that delivers a fixed bolus of additional insulin to compensate for glucose increases.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the disclosure described herein.

It should be appreciated that any of the components or modules referred to with regards to any of the present disclosure embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Additional descriptions of aspects of the present disclosure will now be provided with reference to the accompanying drawings. The drawings form a part hereof and show, by way of illustration, specific embodiments or examples.

### REFERENCES

Previous works explored RL to optimize insulin but were primarily focused on therapy parameters adaptation (Tejedor et al., 2020). Other works focused on adapting the bolus calculator but did not use RL (Unsworth et al., 2023). A few have explored the use of RL to optimize bolus calculators:
(i) (Zhu et al., 2020) proposed a method using double-deep Q-Learning. The RL agent learns to select a percentage to modify the dose given by the standard bolus calculator. This system still relies on standard bolus calculation and has a restricted action space potentially limiting the benefits of deep learning.
(ii) (Ahmad et al., 2022) proposes a method for automatic bolus generation without carbohydrate amounts, and only the meal type is announced (breakfast, lunch, or dinner). The authors reported issues when the meal was unexpectedly small.
(iii) (Jaloli & Cescon, 2023) use Soft-Actor-Critic algorithm to optimize bolus for MDI therapy. Their agent learns to give a bolus based only on glucose levels and meal history, with no notion of the standard bolus calculator, but the boluses are not user-initiated so the system can request a bolus at any time which may increase the system burden.
(iv) (El Fathi & Breton, 2023) proposed a new bolus calculator based on meal categories instead of carbohydrate counting. However, the optimization algorithm needed multiple weeks to converge.
Ahmad, S., Beneyto, A., Contreras, I., & Vehi, J. (2022). Bolus Insulin calculation without meal information. A reinforcement learning approach. Artificial Intelligence in Medicine, 134, 102436.
Brown, S. A., Kovatchev, B. P., Raghinaru, D., Lum, J. W., Buckingham, B. A., Kudva, Y. C., Laffel, L. M., Levy, C. J., Pinsker, J. E., & Wadwa, R. P. (2019). Six-month randomized, multicenter trial of closed-loop control in type 1 diabetes. New England Journal of Medicine, 381(18), 1707-1717.
El Fathi, A., & Breton, M. D. (2023). Using Reinforcement Learning to Simplify Mealtime Insulin Dosing for People with Type 1 Diabetes: In-Silico Experiments. IFAC-PapersOnLine, 56(2), 11539-11544.
Jaloli, M., & Cescon, M. (2023). Reinforcement Learning for Multiple Daily Injection (MDI) Therapy in Type 1 Diabetes (T1D). BioMedInformatics, 3(2), 422-433.
Kovatchev, B. P. (2017). Metrics for glycaemic control—from HbA1c to continuous glucose monitoring. Nature Reviews Endocrinology, 13(7), 425-436.
Schulman, J., Wolski, F., Dhariwal, P., Radford, A., & Klimov, O. (2017). Proximal policy optimization algorithms. ArXiv Preprint ArXiv:1707.06347.
Tejedor, M., Woldaregay, A. Z., & Godtliebsen, F. (2020). Reinforcement learning application in diabetes blood glucose control: A systematic review. Artificial Intelligence in Medicine, 104, 101836.
Unsworth, R., Avari, P., Lett, A. M., Oliver, N., & Reddy, M. (2023). Adaptive bolus calculators for people with type 1 diabetes: A systematic review. Diabetes, Obesity and Metabolism, 25(11), 3103-3113.
Zhu, T., Li, K., Herrero, P., & Georgiou, P. (2020). Basal glucose control in type 1 diabetes using deep reinforcement learning: An in silico validation. IEEE Journal of Biomedical and Health Informatics, 25(4), 1223-1232.

1. A computer implemented method of estimating a universal function for calculating an insulin dose for a subject, the method comprising:
using a computer comprising a processor connected to computer memory storing software to implement computer readable instructions that perform steps comprising:
retrieving raw data of sets comprising a number (N) of observations comprising glucose levels, insulin doses, and carbohydrate intake estimates collected from a population of subjects over a selected time period;
applying the raw data to a reinforcement learning (RL) neural network comprising self-attention subroutines by performing additional steps comprising:
pre-processing the raw data;
segmenting the raw data with a sliding window function;
saving, in the computer memory, a state matrix of the raw data by rearranging segmented raw data to align periodic events within the raw data, identified across the population, as a time series of neural network data;
calculating an encoded state matrix by applying the state matrix to an encoder component of the RL neural network, wherein the encoder component applies at least one self-attention layer to the state matrix;
passing the encoded state matrix to an actor component and a value component programmed as subroutines of the RL neural network;
wherein the actor component defines a function to estimate a current bolus dose of insulin (B_{N}) that is a suggested action to take for a proposed carbohydrate intake (M_{N}); and
wherein the value component assigns a qualitative value to the suggested action by calculating a reward function using a target glucose value and a calculated glucose value that will result from the suggested action;
iteratively evaluating the reward function to maximize the reward function; and
selecting a suggested action corresponding to a maximum reward value as a recommended bolus dose of insulin.

2. The computer implemented method of claim 1, wherein the pre-processing comprises normalizing the raw data and/or applying a decay function to the raw data.

3. The computer implemented method of claim 1, wherein applying at least one self-attention layer to the state matrix comprises saving a last hidden state matrix from the encoder component as the encoded state matrix.

4. The computer implemented method of claim 1, wherein segmenting the raw data comprises saving slices of the data, wherein the slices of the data comprise multiple observations from the raw data corresponding to a window size and a stride size used to segment the raw data.

5. The computer implemented method of claim 4, wherein the slices of the data comprise related observations selected from glucose levels, insulin doses, or carbohydrate intake estimates.

6. The computer implemented method of claim 4, wherein respective slices of related observations are matched as sequence elements, and the sequence elements are combined into a sequence of length (L) having rows that comprise sequence elements comprising the related observations selected from glucose levels, insulin doses, or carbohydrate intake estimates.

7. The computer implemented method of claim 4, wherein the actor component calculates the function, with the sequence in the state matrix of N observations over the time period, B_{N}=f(M^{˜}_{N}, G^{˜}_{N}, H_{1:N-1}), wherein H_{1:N} is the complete data set of the encoded state matrix.

8. The computer implemented method of claim 7, wherein the complete data set H_{1:N} comprises insulin I equal to B_{N}+U_{N}, where B_{N} is an agent suggested action and U_{N} is any additional insulin delivered at t_{N}, including the delivered basal insulin.

9. A computer implemented method of estimating a universal function for calculating an insulin dose for a subject, the method comprising:
using a computer comprising a processor connected to computer memory storing software to implement computer readable instructions that perform steps comprising:
retrieving raw data of sets comprising a number (N) of observations comprising glucose levels and at least one of insulin doses or carbohydrate intake estimates collected from a population of subjects over a selected time period;
applying the raw data to a reinforcement learning (RL) neural network comprising self-attention subroutines by performing additional steps comprising:
pre-processing the raw data;
segmenting the raw data with a sliding window function;
saving, in the computer memory, a state matrix of the raw data by rearranging segmented raw data to align periodic events within the raw data, identified across the population, as a time series of neural network data;
calculating an encoded state matrix by applying the state matrix to an encoder component of the RL neural network, wherein the encoder component applies at least one self-attention layer to the state matrix;
passing the encoded state matrix to an actor component and a value component programmed as subroutines of the RL neural network;
wherein the actor component defines a function to estimate a current bolus dose of insulin (B_{N}) that is a suggested action to take for a subject; and
wherein the value component assigns a qualitative value to the suggested action by calculating a reward function using a target glucose value and a calculated glucose value that will result from the suggested action;
iteratively evaluating the reward function to maximize the reward function; and
selecting a suggested action corresponding to a maximum reward value as a recommended bolus dose of insulin.

10. A computer implemented method according to claim 9, wherein the raw data sets comprise glucose levels and insulin doses in the absence of carbohydrate intake estimates, and the suggested action is application of an automated bolus that delivers selected boluses of additional insulin to compensate for glucose increases.

11. A computer implemented method according to claim 9, wherein the raw data sets comprise glucose levels in the absence of insulin doses and carbohydrate intake estimates, and the suggested action is application of an automated bolus that delivers a fixed bolus of additional insulin to compensate for glucose increases.
